(19) United States
(12) Patent Application Publication
(10) Pub. No.: US 2005/0089952 A1
(43) Pub. Date: Apr. 28, 2005

(54) Apparatuses and processes for increasing protein PEGylation reaction yields

(75) Inventors: Mark D. Chavez, Raleigh, NC (US); Jozef Orpiszewski, Cary, NC (US)

Correspondence Address: AKZO NOBEL PHARMA PATENT DEPARTMENT, PO BOX 318, MILLSBORO, DE, 19966, US

(73) Assignee: Akzo Nobel N.V., Arnhem (NL)

(21) Appl. No.: 10/971,748

(22) Filed: Oct. 21, 2004

Related U.S. Application Data
(60) Provisional application No. 60/513,895, filed on Oct. 22, 2003.
(60) Provisional application No. 60/542,106, filed on Feb. 4, 2004.

(51) Int. Cl. C12P 21/06; C07K 14/47; C08G 63/48; C08G 63/91

(52) U.S. Cl. 435/68.1; 530/350; 525/54.1

(57) ABSTRACT

Embodiments of the present invention generally relate to Apparatuses and Processes for Increasing Protein PEGylation Reaction Yields through repeatable steps of PEGylation and filtration.

### FIELD OF THE INVENTION

Various embodiments of the present invention generally relate to the pegylation of proteins.

### BACKGROUND

The attachment of polyethylene glycol (PEG) to therapeutic proteins has been a successful technique in developing drugs, improving clinical properties such as better physical and thermal stability, protection against proteolysis, increased in vivo circulation half-life, decreased clearance, reduced immunogenicity, antigenicity and toxicity, and enhanced in vivo activity^{A,B,C}. In this manner, PEGylation often improves the safety and efficacy of therapeutics. For example, PEGylation, including, but not limited to, site-specific PEGylation, can improve drug performance by optimizing pharmacokinetics, increasing bioavailability, decreasing immunogenicity and dosing frequency, and/or the like. As PEGylation is employed more frequently, techniques to increase the yield of such reactions become increasingly more valuable.

Covalently attaching the water soluble polymer polyethylene glycol (PEG) to proteins is a prevalent strategy to increase the efficacy of therapeutics¹. Often this is done to alter the circulation half-life by increasing the overall size of the therapeutic entity.

It has been demonstrated that renal clearance occurs with molecules as large as a hemoglobin dimer (32 kDa)⁵ (i.e. blood in urine). Accordingly, to prevent and/or inhibit renal clearance one would design a molecule to have an effective size to be similar to proteins retained in the plasma, i.e. >50 kDa. A typical reaction of the PEG reagent, in this example, and not meant by way of limitation, is an active ester succinimidyl propionate (mPEG-SPA) with nucleophilic sites such as, and not by way of limitation, primary amines on the protein. The reaction is depicted in FIG. 1.

Examples of multiple reactions of the PEG reagent are shown below:
mPEG-SPA+OH⁻→mPEG-OH+free NHS rate constant=k1
mPEG-SPA+protein→PEG-protein+free NHS rate constant=k2
PEG-protein+n (mPEG-SPA)→PEG₍ₙ₊₁₎-protein+n (free NHS) rate constant=k3

The reaction resulting in the monoPEGylated protein species is frequently desired, as additional PEGylation is may decrease activity due to the increased chance of active site blockage with each additional PEG.

Further improvements in the PEGylation reagents are anticipated to further enhance the popularity and applicability of the technology². However, the PEGylation reaction does have fundamental characteristics that limit the utility of the technology. The PEGylation reaction is typically conducted with purified protein, often an expensive and labor intensive endeavor, to minimize the use of PEG reagent and simplify downstream purification of PEGylation byproducts. Additionally, it is difficult to control the extent of PEGylation. For example, the monoPEGylated protein, frequently the target product, can be eliminated by subsequent undesirable PEGylation. Accordingly, the art field desires an improved process that can maximize the use of unreacted protein to form the desired PEGylated protein.

A prior art attempt is disclosed in Size-Exclusion Reaction Chromatography (SERC): A New Technique for Protein PEGylation by Fee, Conan, Department of Materials & Process Engineering, University of Waikato, New Zealand 2001. (hereinafter referred to as the SERC article). The SERC article touts a moving reaction zone that allows for the preferential generation of different size PEGylated products. However, this particular use of size exclusion chromatography is not amenable to large scale manufacturing due to capacity limitations, volume requirements, and the difficulty of regulating the protein PEGylation. Accordingly, the art field is in search of a method and/or process to increase the PEGylation of the target protein in a controllable manner while being usable for large scale manufacturing.

### SUMMARY

In varying embodiments of the present invention, taking advantage of the inherent size increase for PEGylated proteins, novel embodiments of methods of the present invention comprise the PEGylation of a protein and subsequent filtration, comprising the steps of separating the unPEGylated protein from the PEGylated species through the use of ultrafiltration and/or diafiltration. Because the ultrafiltration is largely independent of solution conditions, unPEGylated protein can be recovered from the initial PEGylation solution conditions, such as, for example, and not by way of limitation, the permeate. Thus, subsequent reactions with the recovered unPEGylated species can be readily performed with each successive batch cycle netting additional yield increases. This allows for the optimization of a targeted PEGylated species while maintaining consistent reaction conditions required for cGMP manufacturing.

Therefore, in an embodiment, the present invention comprises apparatuses and processes for the separation of unPEGylated protein from the PEGylated protein.

### BRIEF DESCRIPTION OF THE FIGURES

FIG. 1 is an illustration of protein—PEG reaction.

FIG. 2 is an illustration of an embodiment of PEG batch cycling of the present invention.

FIG. 3 is an illustration of SDS-PAGE analysis of the final permeates (13-fold dilution), final retentates and PEGylation reaction mixes (40-fold dilutions).

FIG. 4 is an illustration of Size Exclusion Chromatography of three lysozyme PEGylation reactions of embodiments of the present invention.

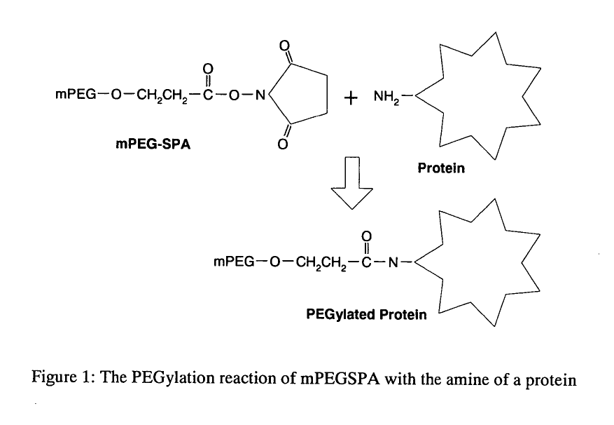

### DETAILED DESCRIPTION

As used herein, the term “PEGylation” means and refers to modifying a protein by covalently attaching polyethylene glycol (PEG) to the protein surface, with “PEGylated” referring to a protein having a PEG attached.

In general, embodiments of the present invention relate to the separation of PEGylated protein from unreacted/unPEGylated protein. In further embodiments, the separation is performed to increase the protein PEGylation reaction yield.

In various embodiments of the present invention, size exclusion chromatography⁶ is used for the separation of unreacted protein from PEGylated protein. In an embodiment, ultrafiltration and/or diafiltration may used to separate/partition the unreacted/unPEGylated protein from the PEGylated protein. In another embodiment, multiple steps of separating are performed the unreacted/unPEGylated protein from the PEGylated protein.

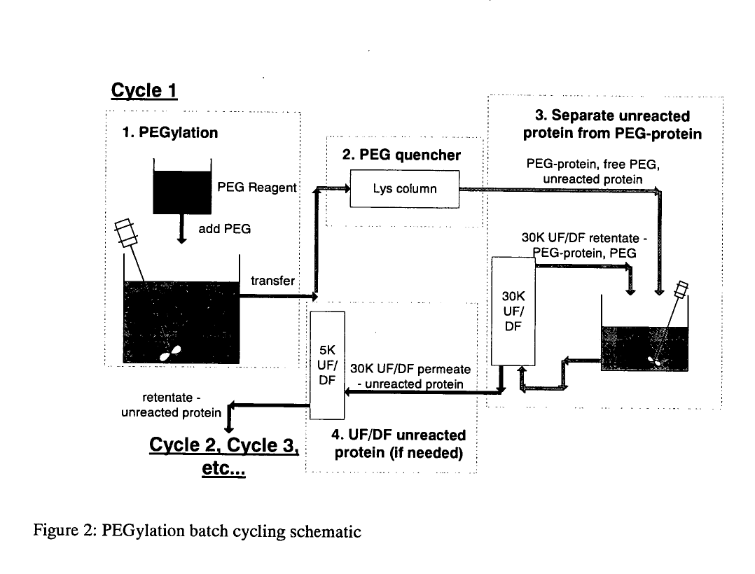

Our invention, in an embodiment, illustrates that separation of unreacted protein from PEGylated protein is achievable using ultrafiltration and/or diafiltration. Since we can accomplish successful partitioning of unreacted protein from PEGylated product, we present an innovative strategy to maximize generation of monoPEGylated proteins through PEGylation batch cycling (See FIG. 2).

In an embodiment, the type of PEG reagent used for the study, mPEG-SPA, was chosen for its reactivity³ and for the stability of the amide linkage generated. However, any PEG reagent may be used with various embodiments of the present invention. For example, and not by way of limitation, conditions for this type of succinimidyl ester to efficiently react with the α-amino group of lysine (pKa 10.5) are pH 8.3, however, other pH conditions are within the scope of the present invention and the pH should not act as a limitation, and likewise protein concentration is not a limitation, with typical values of 5-20 mg/ml⁴.

Accordingly, various embodiments of the present invention comprise processes for separating an unPEGylated protein from a PEGylated species comprising the step of filtering the PEGylated protein mixture, wherein the unPEGylated protein is recovered in the permeate. Other embodiments further comprise the step of diafiltration. In various embodiments, a step of diafiltration can be used to concentrate the recovered unPEGylated protein and/or buffer exchange into the initial PEGylation solution conditions. Further embodiments comprise a subsequent step of separating the unPEGylated protein from a PEGylated species. While other embodiments further comprise at least one further step of filtering. However, various embodiments of the present invention may encompass one or more steps of filtering.

In an embodiment, the protein is lysozyme. However, any protein or therapeutic molecule may be used.

In various embodiments, the percent recovery of the unreacted protein is greater than 30%. In an alternate embodiment, the percent recovery of the unreacted protein is greater than 40%. In an alternate embodiment, the percent recovery of the unreacted protein is greater than 50%. In an alternate embodiment, the percent recovery of the unreacted protein is greater than 65%. However, the percent recovery of the unreacted protein may vary according to the solution conditions, protein properties, type of PEG reagent, PEGylation conditions and the like, as would be within the skill of one of ordinary skill in the art. In this context, the percent recovery of the unreacted protein means and refers to the total percentage of protein that is unPEGylated after the PEGylation reaction.

In various embodiments, the percent yield of the PEGylated protein is greater than 15%. In other various embodiments, the percent yield of the PEGylated protein is greater than 25%. In other various embodiments, the percent yield of the PEGylated protein is greater than 35%.. In other various embodiments, the percent yield of the PEGylated protein is greater than 45%. In other various embodiments, the percent yield of the PEGylated protein is greater than 50%. In other various embodiments, the percent yield of the PEGylated protein is greater than 55%. In other various embodiments, the percent yield of the PEGylated protein is greater than 60%. In other various embodiments, the percent yield of the PEGylated protein is greater than 70%. In other various embodiments, the percent yield of the PEGylated protein is greater than 80%. In other various embodiments, the percent yield of the PEGylated protein is greater than 90%. In other various embodiments, the percent yield of the PEGylated protein is greater than 95%. In other various embodiments, the percent yield of the PEGylated protein is greater than 99%. However, the percent yield of the PEGylated protein may vary. In this context, the percent yield of the PEGylated protein means and refers to the total percentage of the unPEGylated protein that is PEGylated during the reaction and/or reactions.

In various embodiments, there are multiple steps of separating the unPEGylated protein from a PEGylated species. In an embodiment, there are at least two steps of separating the unPEGylated protein from a PEGylated species. In an alternate embodiment, there are at least three steps of separating the unPEGylated protein from a PEGylated species. However, the number of steps of separating the unPEGylated protein from a PEGylated species may vary and can be any number.

In various embodiments, the weight of the protein may vary. In an embodiment, the protein is selected from a protein with a weight of about 0.5 kDa to about 500 kDa. In an alternate embodiment, the protein is selected from a protein with a weight of about 10 kDa to about 300 kDa. In an alternate embodiment, the protein is selected from a protein with a weight of about 25 kDa to about 150 kDa. In an alternate embodiment, the protein is selected from a protein with a weight of about 50 kDa to about 100 kDa. However, any protein may be used with this process and weight is not a limiting factor.

Any protein may be used with differing embodiments of the present invention. In an embodiment, the specie being PEGylated is selected from the group consisting of a protein, enzymes, polypeptide, drugs, dyes, nucleoside, oligonucleotide, lipid, phospholipids, and/or the like. In various embodiments, the PEGylation is performed to enhance the therapeutic value. Examples, and not meant as limitations, of PEGylation to enhance the therapeutic value of a biological molecule are numerous, including but not inclusive of proteins such as single chain Fv single chains^{D}, interferons (U.S. Pat. Nos. 5,382,657, 6,042,822), filgrastim^{F}, hormones^{G}, enzymes^{H} and small drug molecules such as camptothecin^{E}.

In various embodiments, the recovered PEGylated protein is a mixture/combination of multiPEGylated protein and monoPEGylated protein. In various other embodiments, the multiPEGylated protein and/or specie is separated from the monoPEGylated specie/protein. In other embodiments, the multiPEGylated protein is reprocessed into monoPEGylated protein.

While the invention has been described in connection with specific embodiments thereof, it will be understood that it is capable of further modifications and the appended Claims are intended to cover any variations, uses, or adaptations of the invention following, in general, the principles of the invention and including such departures from the present disclosure as come within known or customary practice within the art to which the invention pertains and as may be applied to the essential features hereinbefore set forth whether now existing or after arising. Further, while embodiments of the invention have been described with specific dimensional characteristics and/or measurements, it will be understood that the embodiments are capable of different dimensional characteristics and/or measurements without departing from the principles of the invention and the appended Claims are intended to cover such differences. Furthermore, all patents and other publications mentioned herein are herby incorporated by reference.

### EXAMPLES

### Example 1

Materials and Methods

Procedure

A 150 μM solution of lysozyme (Sigma) was PEGylated with either 5, 20 or 30 kDa mPEG-SPA (Nektar Therapeutics) using a 2:1 PEG:lysozyme molar ratio for 65min at room temperature in 25 mM Sodium Phosphate, pH 8. The reaction was quenched with 1M Glycine to inactivate any PEG molecules that had not already been hydrolyzed. The PEGylated reaction mixture was then to be applied to a tangential flow unit (Minim™ Tangential Flow Filtration System from PALL Corporation) to separate the components based on size. The 30,000 MWCO PES membranes (Vivascience) were new for each experiment and an initial amount of 400 mL of water was flushed through the membrane and out the filtrate to remove preservative from the system. The system was then equilibrated in 25 mM Sodium Phosphate, pH 8 buffer with a feed flowrate set at 90 mL/min to be used across all experiments. Upon equilibration of the system, 100 mL of the PEGylated reaction mixture was added to the retentate vessel and recirculated for 10 min. A transmembrane pressure (TMP) of 10 psi was then set and maintained throughout the run. Once the TMP was set, the system was allowed to stabilize for an additional 10 min in recirculation mode. Samples were pulled from the bulk retentate, retentate line, and permeate line for analysis. The retentate was then concentrated by removing 25 mL through the filtrate (22.5% reduction in bulk retentate volume). The system was again allowed to stabilize at the new concentration for 10min in recirculation mode before samples were taken from the bulk retentate, retentate line, permeate line, and bulk permeate for analysis. Finally, the retentate was concentrated once more by removing an additional 25 mL through the filtrate (29% reduction in bulk retentate volume). The system was again allowed to stabilize at the new concentration for 10 min in recirculation mode before samples were taken from the bulk retentate, retentate line, permeate line, and bulk permeate for analysis. The final retentate volume was removed from the system reservoir and the membrane flushed with 2×15 mL of 25 mM Sodium Phosphate, pH 8 to recover any remaining protein. The samples taken were then analyzed by SEC to observe the sieving across the membrane and if separation by mechanical means was possible using tangential flow filtration.

Analytical Methods

Lysozyme concentration pre-PEGylation was determined by UV detection (ε=2.68). After PEGylation, relative amounts of unreacted lysozyme and PEGylated components were determined by analytical SEC-HPLC using Superose 12 (Amersham Biosciences) with 50 mM Sodium Phosphate, 150 mM NaCl, pH 7 running buffer. SDS-PAGE analysis was carried out on 12% NuPAGE gels with MOPS Running Buffer (Novex) and then stained with Colloidal Blue (Novex).

Results and Discussion

The PEGylation reactions using 5 kDa, 20 kDa and 30 kDa PEG yielded similar results with an average result of 21% unPEGylated lysozyme, 49% monoPEGylated lysozyme, and 30% multiPEGylated lysozyme based on SEC data. Yield would be increased if the unused unPEGylated protein is recycled and PEGylated again. Assuming 100% recovery of the unreacted protein from the first reaction, % monoPEGylated lysozyme can be increased 21% to 58.8% with one additional PEGylation and

TABLE 1
PEGylation Reaction Actual and Projected Yields

| | | | |
|---|---|---|---|
| Reaction | unPEGylated % | monoPEGylated % | multiPEGylated % |

1^{st} Reaction Actual Yields

| | | | |
|---|---|---|---|
| Lysozyme 5 kDa PEG | 23.3 | 44.4 | 32.3 |
| Lysozyme 20 kDa PEG | 18.3 | 49.1 | 32.6 |
| Lysozyme 30 kDa PEG | 22.0 | 52.1 | 25.9 |
| Average => | 21.2 | 48.5 | 30.3 |

2^{nd} Reaction Projected
Yields (recycling unPEGylated lysozyme from reaction 1)*

| | | | |
|---|---|---|---|
| Lysozyme 5 kDa PEG | 5.4 | 54.7 | 39.8 |
| Lysozyme 20 kDa PEG | 3.3 | 58.1 | 38.6 |
| Lysozyme 30 kDa PEG | 4.9 | 63.5 | 31.6 |
| Average => | 4.5 | 58.8 | 36.7 |

3^{rd} Reaction Projected
Yields (recycling unPEGylated lysozyme from reaction 2)*

| | | | |
|---|---|---|---|
| Lysozyme 5 kDa PEG | 1.3 | 57.1 | 41.6 |
| Lysozyme 20 kDa PEG | 0.6 | 59.7 | 39.7 |
| Lysozyme 30 kDa PEG | 1.1 | 66.1 | 32.9 |
| Average => | 1.0 | 61.0 | 38.1 |

*Estimated yields

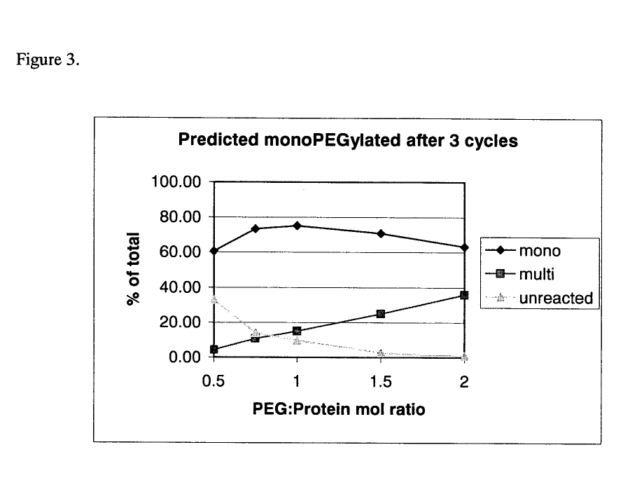

increased 26% overall to 61.0% with two additional PEGylations. See Table 1 and FIG. 3 for breakdown of PEGylation reaction actual and projected yields.

To achieve projected increases in yield, successful separation of unreacted protein from PEGylated protein must be accomplished. To achieve this goal, the resulting PEGylation mixtures from reacting 5 kDa, 20 kDa, and 30 kDa with lysozyme were applied over a 30,000 MWCO membrane using tangential flow. Of note, a 30,000 MWCO membrane is a reasonable model for kidney function, hence potential products would most preferably be retained by this membrane.

The molecular weight of lysozyme is approximately 14,300 Da. When conjugating PEG to lysozyme, the molecular weight will increase to give actual molecular weights of 19,300 Da (5 kDa PEG), 34,300 Da (20 kDa PEG), and 44,300 Da (30 kDa PEG) for the new monoPEGylated molecules and greater for any multiPEGylated species. These new molecules, however, tended to exhibit larger sizes than the additive weights of their individual components due to the properties of PEG. When the monoPEGylated species were analyzed via SDS-PAGE, the observed sizes corresponded to 22 kDa, 52 kDa, and 64 kDa respectively (See FIG. 3). When the same species were analyzed via SEC-HPLC, the observed sizes corresponded to 37 kDa, 256 kDa, and an estimated 426 kDa respectively (See FIG. 4) relative to gel filtration standards (Bio-Rad). The larger apparent size invoked by the PEG is due to the extensive water structure associated with the PEG, adding effective size and bulk.

With the PEGylation reaction conducted with different sized PEGs (5 kDa, 20 kDa, 30 kDa), based on the actual molecular weights, we predicted acceptable unPEGylated lysozyme passage through the 30 kDA membrane. For the other components in each PEGylation reaction mixture, we predict some passage of the 5 kDa PEG-lysozyme monoPEGylated species, little of the 20 kDa PEG-lysozyme monoPEGylated species and very little of any other PEGylated species. Indeed, these expectations fit the data obtained. During the concentration, unreacted lysozyme passed through the membranes at acceptable levels regardless of the PEG size, 21% (5 kDa PEG-lysozyme), 36% (20 kDa PEG-lysozyme), and 24% (30 kDa PEG-lysozyme) of total unreacted lysozyme in each experiment passed through the membrane. When looking at the passage of PEGylated molecules, monoPEGylated species of 5 kDa PEG-lysozyme passed through the membranes at only 3% of total and multiPEGylated species passage was at 0.2% of total. MonoPEGylated species of 20 kDa PEG-lysozyme passed through the membranes at only 4% of total and there was no multiPEGylated species passage. And no PEGylated species of the 30 kDa PEG-lysozyme species passed through the membrane. The excellent separation obtained between the unreacted species (lysozyme) and PEGylated species (worst case=88% unreacted lysozyme) allows for efficient preferential recovery of the unreacted species that is now available for further PEGylation while retaining the majority of the PEGylated species.

When taking a closer look at sieving (% protein passage through the membrane) of the different species through the membrane, sieving was always highest at the lowest concentrations and decreased as the concentration increased. This is not unexpected, as a gel layer will form on the membrane surface as backpressure is applied to the retentate to generate a higher permeate flow rate. As concentrations increase, this gel layer should increase in size and allow fewer particles to pass through the membrane. At the highest concentration, sieving of lysozyme through the 30kDa membrane was detected to be 35% (5 kDa PEG-lysozyme), 73% (20 kDa PEG-lysozyme), and 42% (30 kDa PEG-lysozyme). In all cases, the difference in sieving between the unreacted protein (>35%) and the PEGylated protein (<4%) confirms effective partitioning and the potential to increase the overall PEGylation yield through recovery of the unreacted protein.

### Example 2

Purpose: To increase the overall PEGylation yield by recovering the unreacted protein and performing subsequent PEGylation reactions

Procedure:

1. Characterization of PEGylation Reaction

The PEG:protein molar ratio was varied to characterize the production of monoPEGylated and multiPEGylated species. The reaction conditions were 25 mM phosphate pH 8.0, 2.15 mg lysozyme/ml, at room temperature for 1 hour. 10% w/v PEG (30,000 MW) solution was added at varied amounts to generate different mol ratio. The results as measured by SEC-HPLC of the PEGylation reactions with different PEG:protein mol ratios are illustrated in the table below:

TABLE 2
Distribution of PEGylation species as a function of mol ratio

| PEG:protein mol ratio | % unreacted | % mono | % multi |
|---|---|---|---|
| 2 to 1 | 20 | 51 | 29 |
| 1.5 to 1 | 30 | 51 | 18 |
| 1 to 1 | 46 | 45 | 9 |
| 0.75 to 1 | 52 | 41 | 6 |
| 0.5 to 1 | 69 | 28 | 2 |

The maximum amount of monoPEGylated material generated was 51%. As seen in the 2:1 mol ratio, additional amounts of PEG results in an overall conversion of unreacted lysozyme to multiPEGylated, with the amount of monoPEGylated protein remaining static. As seen in the 2:1 and 1.5:1 mol ratios, additional PEG depleted the unreacted lysozyme, resulting in generation of multiPEGylated species at the expense of monoPEGylated protein.

For an ideal PEG batch cycling experiment, conditions are such that significant amounts of monoPEGylated lysozyme is generated while minimizing the amount of multiPEGylated species. From the single batch experiments above, one can predict the generation of monoPEGylated lysozyme for a specified number of cycles. Assuming 100% recovery of the unreacted lysozyme, the predicted monoPEGylated amounts for three batch cycles is presented in FIG. 5:

A plateau is reached at about 1:1 PEG:Protein mol ratio, thus this ratio was used for the experiment.

2. Batch Cycle 1

The reaction conditions were 25 mM phosphate pH 8.0, 2.15 mg lysozyme/ml, at room temperature for 1 hour. 10% w/v PEG (30,000 MW) solution was added to generate a PEG:protein mol ratio of 1:1. The results as measured by SEC-HPLC of the PEGylation reaction is illustrated in the table below:

First Reaction: total

| mg/mL | mL rxn | mg Lys | % mono | mg mono | % unreacted | % multi |
|---|---|---|---|---|---|---|
| 2.15 | 300 | 645 | 44.6 | 288 | 46.9 | 8.5 |

The mixture was concentrated with a Pall Minim UF/DF system with a 30,000 MW PES membrane to 100 mL and diafiltered with 4 volumes (400 mL) of reaction buffer (25 mM phosphate pH 8.0). The permeate was collected and concentrated with a 5,000 MW PES membrane approximately 12× and diafiltered with a 5× volume to generate the equivalent reaction buffer conditions as in batch cycle 1. The results are displayed below showing an 88% recovery of the unreacted lysozyme.

Recovery of Unreacted:

unreacted postUF/DF

| | | | | % |
|---|---|---|---|---|
| mg, rxn 1 | mg/mL | mL | mg | recovery |
| 303 | 4.68 | 57 | 267 | 88 |

3. Batch Cycle 2

The reaction conditions were 25 mM phosphate pH 8.0, 2.15 mg lysozyme/ml, at room temperature for 1 hour. 10% w/v PEG (30,000 MW) solution was added to generate a PEG:protein mol ratio of 1:1. The results as measured by SEC-HPLC of the PEGylation reaction is illustrated in the table below:

Recovered Unreacted Material and Second

Reaction:

| mg/mL | mL rxn | mg Lys | % mono | mg mono | % unreacted | % multi |
|---|---|---|---|---|---|---|
| 4.68 | 57 | 267 | 45.1 | 120 | 46.1 | 8.8 |

The mixture was concentrated with a Pall Minim UF/DF system with a 30,000 MW regenerated cellulose membrane and diafiltered with 4 volumes (400 mL) of reaction buffer (25 mM phosphate pH 8.0). The permeate collected showed leakage of PEGylated species, indicating that the RC membrane did not separate unreacted lysozyme from PEGylated lysozyme as efficiently as the PES membrane. Thus, the retentate and permeate were pooled together and reconcentrated with a 5,000 MW PES membrane. This mixture was then place onto the 30,000 MW PES membrane, concentrated, and diafiltered accordingly. The permeate collected containing the isolated lysozyme was then concentrated with the 5K PES membrane and diafiltered with a 5× volume to generate the equivalent reaction buffer conditions as in batch cycle 1. The results are displayed below showing a 47% recovery of the unreacted lysozyme. The losses are likely due to the multiple UF/DF steps required as a result of the poor RC membrane performance.

Recovery of Unreacted:

unreacted postUF/DF

| | | | | % |
|---|---|---|---|---|
| mg, rxn 2 | mg/mL | mL | mg | recovery |
| 123 | 4.68 | 57 | 58 | 47 |

4. Batch Cycle 3

The reaction conditions were 25 mM phosphate pH 8.0, 2.15 mg lysozyme/ml, at room temperature for 1 hour. 10% w/v PEG (30,000 MW) solution was added to generate a PEG:protein mol ratio of 1:1. The results as measured by SEC-HPLC of the PEGylation reaction is illustrated in the table below:

Recovered Unreacted Material and Third Reaction:

| mg/mL | mL rxn | mg Lys | % mono | mg mono | % unreacted | % multi |
|---|---|---|---|---|---|---|
| 2 | 28.71 | 57 | 44 | 25 | 47.4 | 8.5 |

5. Overall Results

The results of PEG batch cycling is summarized in the table below:

Yields:

| | % yield |
|---|---|
| Overall | 67.2 **Max. yield for single cycle: |
| | 51% |
| 1st rxn | 44.6 |
| 2nd rxn | 18.7 |
| 3rd rxn | 3.9 |

The theoretical yield predicted was 75%, whereas the actual yield was 67.2%. Losses were mainly attributable to the incomplete recovery of unreacted lysozyme. PEG batch cycling for this experiment resulted in an increase of monoPEGylated lysozyme from 51% of the starting material to 67% of the starting material.

References

¹Harris, J. M., and Zalipsky, S. (eds.) “Poly(ethyleneglycol): Chemistry and Biological Applications”, ACS Books, Washington, D.C. 1997.
²Kozlowski, A., and Harris, J. M. “Improvements in Protein PEGylation: PEGylated interferons for treatment of hepatitis C”, J. Controlled Release 72 (2001), 217-224.
³Harris, J. M., and Kozlowski, A. “Poly(ethylene glycol) and Related polymers Monosubstituted with Propionic or Butanoic Acids and Functional Derivatives Thereof for Biotechnical Applications”, U.S. Pat. No. 5,672,662 1997.
⁴“Amine-Reactive Probes”, Molecular Probes, Inc. Feb. 25, 2002
⁵Winslow, R. W. “Blood Substitutes: Physiological Basis of Efficacy”, Birkhauser Verlag, 1995.
⁶Fee, C. J. “Size-Exclusion Reaction Chromatography (SERC): A New Technique for Protein Pegylation”, Biotechnology and Bioengineering 82:2 (2003), 200-206.
^{A}Bailon P, and Berthold W. “Polyethylene glycol-conjugated pharmaceutical proteins”, Pharm Sci Technol Today 1 (1998), 352-356.
^{B}Yowell S. L., and Blackwell S. “Novel effects with polyethylene glycol modified pharmaceuticals”, Cancer Treat Rev 28 (2002), 3-6.
^{C}Mehvar R. “Modulation of the pharmacokinetics and pharmacodynamics of proteins by polyethylene glycol conjugation”, J Pharm Sci 3 (2000), 125-136.
^{D}Lee L S, Conover C, Shi C, Whitlow M, Filpula D. “Prolonged circulating lives of single-chain Fv proteins conjugated with polyethylene glycol: a comparison of conjugation chemistries and compounds”, Bioconjug Chem. 10(6) (1999), 973-81.
^{E}Conover C D, Greenwald R B, Pendri A, Gilbert C W, Shum K L. “Camptothecin delivery systems: enhanced efficacy and tumor accumulation of camptothecin following its conjugation to polyethylene glycol via a glycine linker”, Cancer Chemother Pharmacol. 42(5) (1998), 407-14.
^{F}Crawford J. “Pegfilgrastim administered once per cycle reduces incidence of chemotherapy-induced neutropenia”, Drugs. 62 Suppl 1 (2002), 89-98
^{G}Parkinson C, Scarlett J A, Trainer P J. “Pegvisomant in the treatment of acromegaly”, Adv Drug Deliv Rev. 55(10) (2003), 1303-14
^{H}Ellis K M, Mazzoni L, Fozard J R. “Role of endogenous adenosine in the acute and late response to allergen challenge in actively sensitized Brown Norway rats”, Br J Pharmacol. 139(6) (2003), 1212-8.

1. A process for increasing protein PEGylation reaction yields comprising the steps of:
i. separating an unPEGylated protein from a PEGylated species comprising the step of filtering the PEGylated protein mixture, wherein the unPEGylated protein is recovered through the permeate in initial PEGylation solution conditions and
ii. repeating the step of separating.

2. The process of claim 1 wherein the step of separating comprises the step of diafiltration.

3. The process of claim 1 further comprising at least one subsequent step of separating the unPEGylated protein from a PEGylated species.

4. The process of claim 3 further comprising a second step of filtering.

5. The process of claim 1 further comprising the step of PEGylating an unPEGylated protein.

6. The process of claim 3 further comprising the step of PEGylating an unPEGylated protein.

7. The process of claim 6 wherein at least three steps of separating the unPEGylated protein from a PEGylated species are performed.

8. The process of claim 1 wherein the PEGylated protein separated is substantially monoPEGylated protein.

9. The process of claim 8 further comprising the step of separating multiPEGylated from monoPEGylated protein.

10. The process of claim 9 wherein the multiPEGylated protein is reprocessed into monoPEGylated protein.

11. The process of claim 4 wherein the second step of filtering is diafiltration.

12. A process for recovering PEGylated protein from a PEGylation reaction of unPEGylated protein comprising the steps of:
i. PEGylating a protein through a PEGylation reaction and
ii. filtering the PEGylated protein, wherein the unPEGylated protein is recovered in initial PEGylation solution conditions.

13. The process of claim 12 wherein the unPEGylated protein is recovered in the permeate.

14. The process of claim 12 wherein the step of filtering comprises ultrafiltration and/or diafiltration.

15. The process of claim 12 wherein the process is repeated.
